(19) United States
(12) Patent Application Publication
(10) Pub. No.: US 2013/0085528 A1
(43) Pub. Date: Apr. 4, 2013

(54) KNOTLESS SUTURE ANCHOR

(71) Applicants: Kristian DiMatteo, Raynham, MA (US); Arthur G. Stephen, Raynham, MA (US); Gregory R. Whittaker, Raynham, MA (US); Mark Capobianco, Raynham, MA (US)

(72) Inventors: Kristian DiMatteo, Raynham, MA (US); Arthur G. Stephen, Raynham, MA (US); Gregory R. Whittaker, Raynham, MA (US); Mark Capobianco, Raynham, MA (US)

(73) Assignee: DePuy Mitek, Inc., Raynham, MA (US)

(21) Appl. No.: 13/249,941

(22) Filed: Sep. 30, 2011

(51) Int. Cl. A61B 17/04

(52) U.S. Cl. 606/232

(57) ABSTRACT

A suture anchor comprises a distal anchor body which can be placed into bone and has a suture leading therefrom to adjacent soft tissue and a proximal anchor body. After placement of the distal anchor body the suture is tensioned and the proximal anchor body is then driven into the bone behind the distal anchor body to lock the suture in a knotless technique.

### BACKGROUND

This application relates to suture anchors and more particularly to knotless suture anchors.

Suture anchors are commonly employed to attach soft tissue such as tendons or ligaments to bone. For instance, in a rotator cuff repair suture is passed through a detached or damaged portion of a rotator cuff tendon. A suture anchor is implanted into the adjacent bone. By attaching the suture to the anchor the tendon is pulled into contact with the bone to promote adhesion of the tendon to the bone.

Such procedures are often performed arthroscopically through a narrow cannula. This reduces trauma to the patient but makes attachment of the suture to the anchor using a knot more difficult. Knotless suture anchors may be employed which allow a surgeon to tension the suture to a desired degree and then affix to suture to the anchor without having to tie a knot. A typical knotless anchor is shown in US Patent Publication No. 20080033460 wherein the suture is trapped between an inner member and outer member of an anchor in coaxial relation to one another. While such anchors work well their complexity increases manufacturing cost and makes it difficult to form the anchor of bioabsorbable materials which often are more frangible and less strong than metals or traditional polymers.

### SUMMARY OF THE INVENTION

The present invention overcomes these and other limitations of the prior art in a simple and elegant design.

A suture anchor system according to the present invention comprises a distal anchor body having a suture attachment and a guide wire trailing proximally from the distal anchor body and a proximal anchor body having a central bore passing therethrough sized to accommodate the guide wire, and one or more purchase enhancements extending from the proximal anchor body.

Preferably, a suture passer comprising an elongated flexible member is threaded through the suture attachment and has a distal suture capture configuration such as a loop through which suture can be passed. Preferably, the distal anchor body is mounted to a first driver having an elongated driver body with the distal anchor body attached to a distal end and the guide wire extending along the driver body.

Preferably, the one or more purchase enhancements comprise screw threads.

A suture anchor system according to the present invention comprises a distal anchor body having a suture attachment and a distal awl tip. The distal anchor body is mounted on an awl driver whereby it can be driven into a bone to create a bone hole. A proximal anchor body is sized to fit into a bone hole created by the distal anchor body and having one or more purchase enhancements extending from the proximal anchor body.

A suture passer comprising an elongated flexible member having a distal suture capture configuration can be threaded through the suture attachment. Preferably, the suture capture configuration comprises a loop through which suture can be passed. Preferably, the purchase enhancements comprise screw threads.

A method according to the present invention provides for attaching a soft tissue to a bone. The method comprises the steps of: implanting into the bone a distal suture anchor body, leaving a guide wire extending proximally out of the bone from the distal suture anchor body; threading a suture from the soft tissue through a suture attachment on the distal suture anchor body, the suture extending from the soft tissue to the distal suture anchor body and out of the bone when the distal suture anchor body is implanted into the bone; and implanting over the guide wire and into the bone a proximal suture anchor body such that at least a portion of the suture is trapped between the proximal suture anchor body and the bone.

Preferably, step of threading the suture through the suture attachment on the distal body is accomplished by placing the suture into a suture capture section of a suture shuttle threaded through the suture attachment and then pulling the suture shuttle through the suture attachment to draw the suture capture section and thus the suture through the suture attachment. Preferably, the suture shuttle is positioned through the suture attachment during the step of implanting the distal anchor body.

Preferably, the proximal anchor body is threaded and the step of implanting the proximal anchor body comprises threading it into the bone behind the distal suture body. Preferably, a desired tension is applied to the suture after implanting the distal anchor body and prior to locking the suture by fully implanting the proximal anchor body.

A method according to the present invention provides for attaching a soft tissue to a bone. The method comprises the steps of: piercing a bone and creating a bone hole with a distal suture anchor body which has a distal awl tip and a suture attachment with a length of suture passing therethrough, the distal suture anchor body being mounted on an awl driver; removing the awl driver and leaving the distal anchor body implanted into the bone hole with a first portion of the suture extending out of the bone hole; threading a second portion of the suture through the soft tissue; tensioning the suture by drawing on the first portion; and implanting into the bone hole proximal of the distal anchor body a proximal suture anchor body such that at least a portion of the suture is trapped between the proximal suture anchor body and the bone.

Preferably, the proximal anchor body is threaded and the step of implanting the proximal anchor body comprises threading it into the bone behind the distal suture body.

### BRIEF DESCRIPTION OF THE DRAWINGS

FIG. 1 is a side elevation view of a suture anchor according to the present invention;

FIG. 2 is a side elevation view in cross-section of a tendon, adjacent bone and the anchor of FIG. 1, specifically a distal anchor body thereof in position for entry into a bone hole;

FIG. 3 is a side elevation view in cross-section of a tendon, adjacent bone and the anchor of FIG. 1, specifically the distal anchor body after placement into the bone hole;

FIG. 4 is a side elevation view in cross-section of a tendon, adjacent bone and the anchor of FIG. 1, specifically suture from the tendon being loaded into a suture shuttle threaded through the distal anchor body;

FIG. 5 is a side elevation view in cross-section of a tendon, adjacent bone and the anchor of FIG. 1, specifically the suture from the tendon loaded into the distal anchor body after shuttling via the suture shuttle;

FIG. 6 is a side elevation view in cross-section of a tendon, adjacent bone and the anchor of FIG. 1, specifically the suture being tensioned;

FIG. 7 is a side elevation view in cross-section of a tendon, adjacent bone and the anchor of FIG. 1, specifically a proximal anchor body positioned for entry into the bone hole behind the distal anchor body;

FIG. 8 is a side elevation view in cross-section of a tendon, adjacent bone and the anchor of FIG. 1, specifically the distal anchor being fully inserted into the bone hole;

FIG. 9 is a side elevation view in cross-section of a tendon, adjacent bone and the anchor of FIG. 1, showing the completed deployment of the anchor into the bone;

FIG. 10 is a side elevation view in cross-section of a tendon, adjacent bone and a further embodiment of a suture anchor according to the present invention, specifically a distal anchor body thereof in position for entry into a bone hole;

FIG. 11 is a side elevation view in cross-section of a tendon, adjacent bone and the anchor of FIG. 10, specifically the distal anchor body being driven into the bone;

FIG. 12 is a side elevation view in cross-section of a tendon, adjacent bone and the anchor of FIG. 10, specifically the suture being tensioned;

FIG. 13 is a side elevation view in cross-section of a tendon, adjacent bone and the anchor of FIG. 10, showing the distal anchor body in place in the bone and the suture under tension;

FIG. 14 is a side elevation view in cross-section of a tendon, adjacent bone and the anchor of FIG. 10, specifically a proximal anchor body in position to enter the bone behind the distal anchor body;

FIG. 15 is a side elevation view in cross-section of a tendon, adjacent bone and the anchor of FIG. 10, specifically the second anchor body being fully driven into the bone; and

FIG. 16 is a side elevation view in cross-section of a tendon, adjacent bone and the anchor of FIG. 10, showing the completed deployment of the anchor into the bone.

### DETAILED DESCRIPTION

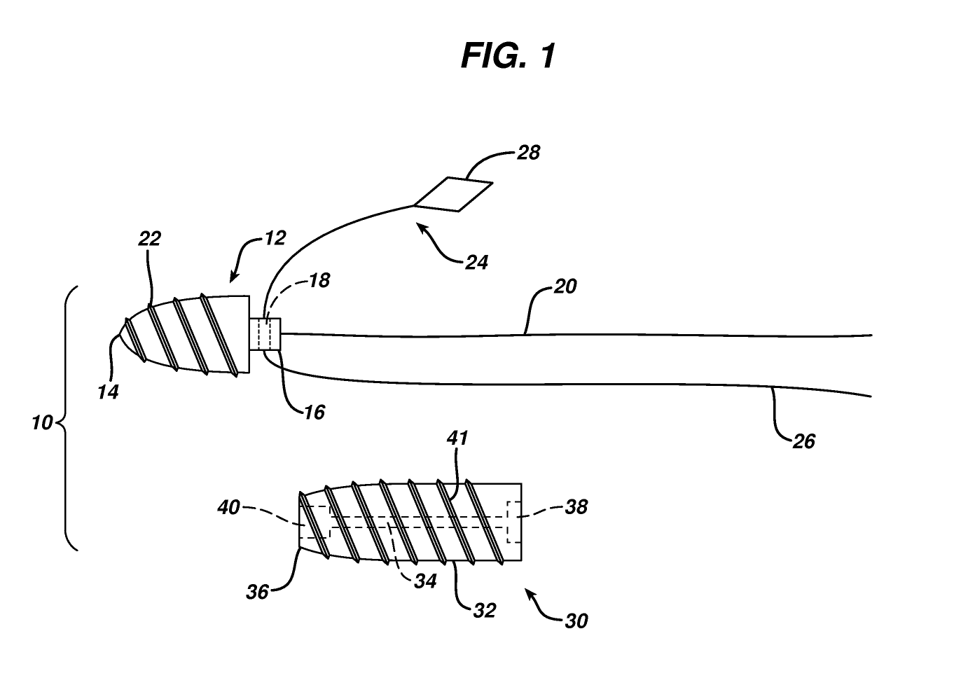

FIG. 1 depicts a knotless suture anchor 10 according to the present invention. It comprises a distal anchor body 12 having a pointed distal tip 14 and a proximally extending extension 16. The extension 16 has a shape, such as hexagonal, for engagement with a driver tool (not shown in FIG. 1) and a suture aperture 18 therethrough for receiving suture (also not shown in FIG. 1). A guide wire 20 extends proximally from the anchor body 12 and is releasably connected thereto, such as by friction or threading. Threads 22 on the body 12 allow for threading of the body 12 into bone. Alternatively, the distal anchor body 12 can be driven awl like into bone in which event the threads 22 can be used for removal or can be omitted altogether. A suture passer 24, such as the CHIA PERCPASSER® available from DePuy Mitek, Inc. of Raynham, Mass., is positioned within the suture aperture 18. It comprises an elongated braided NITINOL wire 26 having a kite shaped distal suture capture loop 28.

The suture anchor 10 further comprises a proximal anchor body 30 having external thread 32 and a central axial bore 34 sized to slidably accommodate the guide wire 20. It has a rounded distal end 36 and a proximal driving tool receiving recess 38, preferably hexagonal. It can also be provided with an axial distal socket 40 sized to accommodate the extension 16 of the distal anchor body 12 to allow the proximal anchor body 30 to advance more deeply into a common bone hole as will be described. Preferably, an additional proximal thread start 41 is provided, having the same pitch as the thread 32, to provide additional holding in harder cortical bone material such as described is U.S. Pat. No. 7,322,978 to West, Jr. which is incorporated herein by reference.

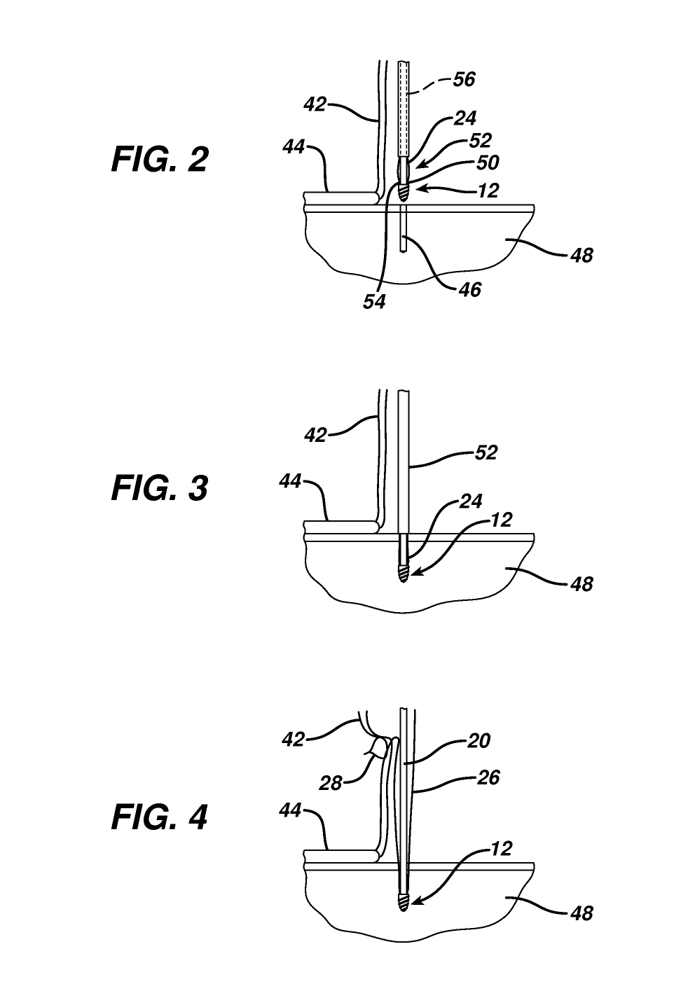

FIGS. 2 to 9 illustrate use of the suture anchor 10 of FIG. 1. A length of suture 42 is passed through a section of tissue 44, such as a tendon in a rotator cuff, and a bone hole 46 is formed in an adjacent bone 48 at a desired location for placement of the anchor 10. The distal anchor body 12, which is disposed on a distal end 50 of a driver 52, is positioned at the bone hole 46 (FIG. 2). The driver 52 has a distal tool end 54 which mates with the proximal extension 16 of the distal anchor body 12 and has one or more axial lumens 56 or grooves for receiving the wire 26. The distal anchor body 12 is then driven into the bone hole 46 (FIG. 3). The driver 52 is removed, leaving the distal anchor 12 in the bone hole 46 with the guide wire 20 extending proximally out of the bone hole 46.

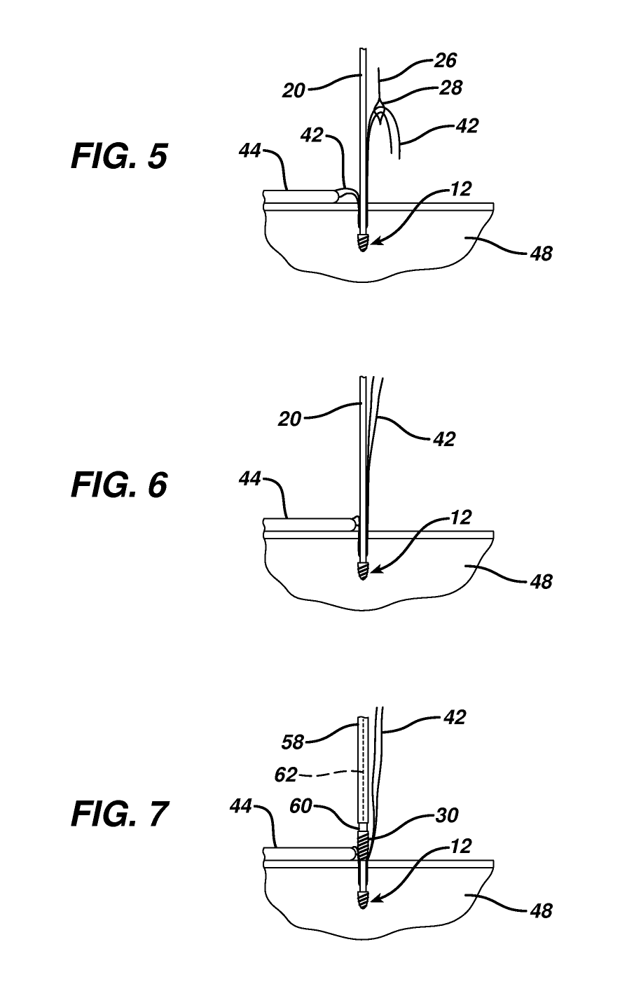

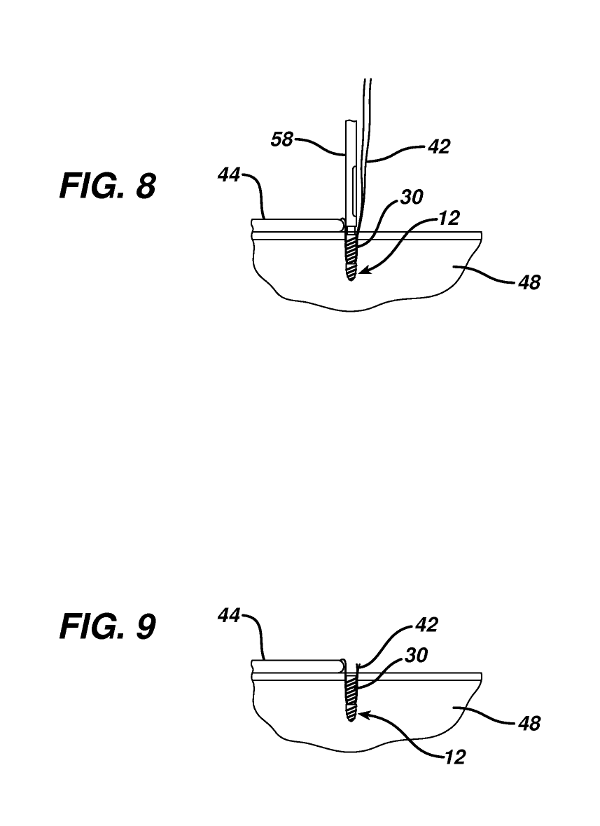

In a preferred arthroscopic procedure, ends of the suture 42, the guide wire 20, the suture capture loop 28 and the other end of the wire 26 all would be extending outside of the patient where the suture 42 would then be loaded into the capture loop 28 (FIG. 4). By drawing on the wire 26 the suture 42 is shuttled through the suture hole 18 of the distal anchor body 12 (FIG. 5). The suture 42 is tensioned to provide the desired position of and tension upon the tissue 44 (FIG. 6). The proximal anchor body 30 is passed distally over the guide wire 20 and positioned at the bone hole 46 (FIG. 7). It is received upon a second driver 58 having a distal tool end 60 which mates with the tool recess 38 of the proximal anchor body 30 and has an axial lumen 62 or groove for receipt of the guide wire 20. Final tension adjustment of the suture 42 can be performed at this time and then the proximal anchor body 30 is driven into the bone hole 46 to lock the suture 42 (FIG. 8). The second driver 58 and the guide wire 20 are removed and the excess suture is 42 trimmed (FIG. 9).

The socket 40 allows the proximal anchor body 30 to be inserted further into the bone hole 30 as its path is not blocked by the extension 16 of the distal anchor body 12. The suture 42 is trapped between the proximal anchor body 30 and the bone hole 46 to lock the suture, and if the proximal anchor body 30 is driven all the way into contract with the distal anchor body 12, with the extension 16 received within the socket 40, the suture 42 can also be trapped between the distal anchor body 12 and proximal anchor body 30 for additional fixation. Optionally, the extension 16 and socket 40 can be configured for engagement to enable additional rotation and further advancement into the bone of the distal anchor body 12.

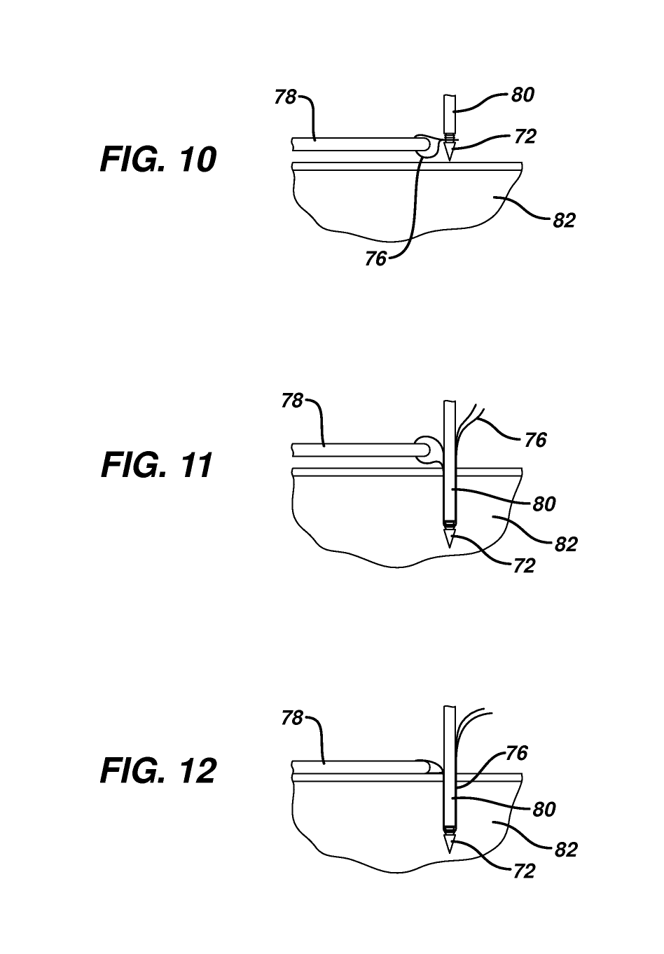

FIGS. 10 to 16 illustrate use of a push-in type suture anchor 70 according to the present invention similar to the suture anchor 10 with a distal anchor body 72 and proximal anchor body 74, but without threading on the distal anchor body 72, a guide wire or a bore through the proximal anchor 74 for a guide wire. Omitting the requirement for a pre-prepared hole minimizes steps to reduce potential error and decrease surgery time.

First, a length of suture 76 is passed through a tissue 78 and is loaded onto the distal anchor body 72. The distal anchor body 72 is loaded onto a driver 80 to form an awl in which the distal anchor body 72 acts as a removable awl tip, and the distal anchor body 72 is positioned over a bone 82 at a desired insertion location (FIG. 10).

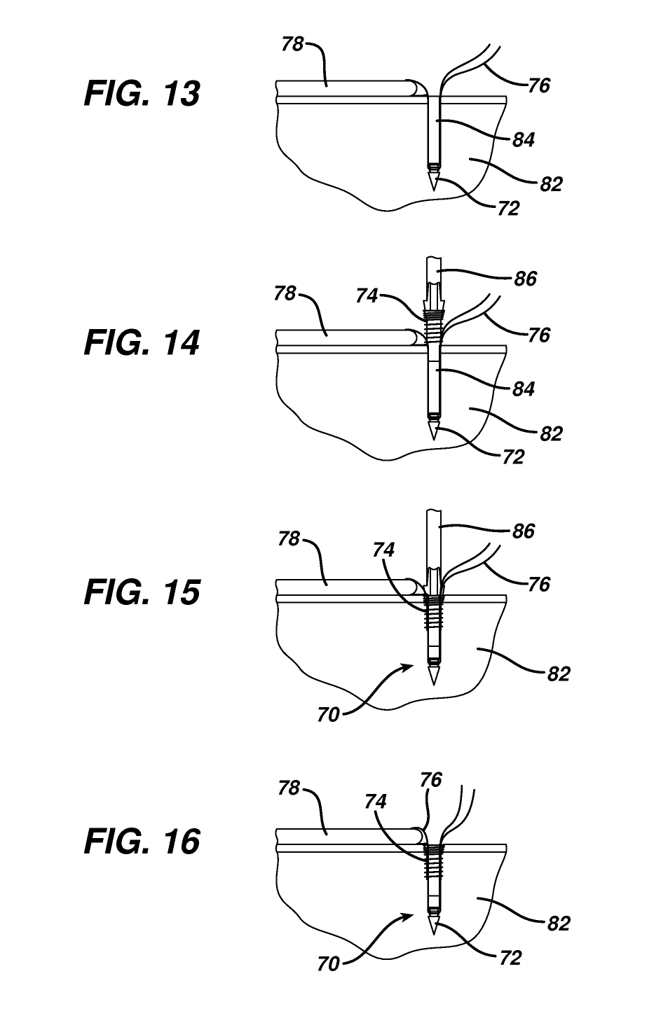

The distal anchor body 72 is then driven into the bone 82, such as by hammering the driver 80 (FIG. 11). The suture 76 is tensioned to provided the desired tension on the tissue 78 (FIG. 12) and the driver 80 is removed leaving the distal anchor body 72 in place at the bottom of a bone hole 84 created by its insertion (FIG. 13). While tension is maintained on the suture 76 the proximal anchor body 74, loaded onto a second driver 86, is inserted into the bone hole 84 (FIG. 14) and is fully threaded in to lock the suture 76 into place (FIG. 15). The second driver 86 is removed and excess suture 76 trimmed to complete the procedure (FIG. 15).

The FIGS. show a simple suturing technique with a single anchor and a simple loop of suture through tissue in order to focus on the operation of the suture anchors 10 and 70. However, the suture anchors 10 and 70 of the present invention have utility in more complex configurations such as a dual-row anchoring with a mattress stitch suture configuration, where a row of anchors (often not knotless anchors such as 10 or 70) is placed under the tendon and a second row of knotless anchors is placed lateral of the tendon with suture extending up from the first row, through the tendon and to the second row in a crossing pattern whereby to hold the tendon down to the bone.

The novel suture anchors of the present invention may be made from a number of suitable materials including a metallic material, a non-biodegradable polymer, a biodegradable polymer, or a composite of a biodegradable polymer or copolymer and a bioceramic. The term biodegradable as used herein is defined to mean materials that degrade in the body and then are either absorbed into or excreted from the body. The term bioceramic as defined herein is defined to mean ceramic and glass materials that are compatible with body tissue. The bioceramics are preferably biodegradable.

The metallic materials that can be used to manufacture the anchors of the present invention include stainless steel, titanium, alloys of nickel and titanium, or other biocompatible metallic materials.

The non-biodegradable materials that can be used to manufacture the anchors of the present invention include polyethylene, polypropylene, PEEK (polyetheretherketone), or other biocompatible non-absorbable polymers.

The biodegradable polymers that can be used to manufacture the anchors used in the present invention include biodegradable polymers selected from the group consisting of aliphatic polyesters, polyorthoesters, polyanhydrides, polycarbonates, polyurethanes, polyamides and polyalkylene oxides. Preferably, the biodegradable polymers are aliphatic polyester polymers and copolymers, and blends thereof. The aliphatic polyesters are typically synthesized in a ring opening polymerization. Suitable monomers include but are not limited to lactic acid, lactide (including L-, D-, meso and D,L mixtures), glycolic acid, glycolide, .epsilon.-caprolactone, p-dioxanone (1,4-dioxan-2-one), trimethylene carbonate (1,3-dioxan-2-one), .delta.-valerolactone, and combinations thereof.

The bioceramics that can be used in the composite anchors of the present invention include ceramics comprising mono-, di-, tri-, .alpha.-tri-, .beta.-tri-, and tetra-calcium phosphate, hydroxyapatite, calcium sulfates, calcium oxides, calcium carbonates, magnesium calcium phosphates. It is particularly preferred to use a .beta.-tritricalcium phosphate. In addition to bioceramics, bioglasses may also be used in the composite screws. The bioglasses may include phosphate glasses and bioglasses.

Suitable biocompatible synthetic polymers can include polymers selected from the group consisting of aliphatic polyesters, poly(amino acids), copoly(ether-esters), polyalkylene oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, polyurethanes, poly(ether urethanes), poly(ester urethanes), polypropylene fumarate), poly(hydroxyalkanoate) and blends thereof.

For the purpose of this invention aliphatic polyesters include, but are not limited to, homopolymers and copolymers of lactide (which includes lactic acid, D-,L- and meso lactide); glycolide (including glycolic acid); .epsilon.-caprolactone; p-dioxanone (1,4-dioxan-2-one); trimethylene carbonate (1,3-dioxan-2-one); alkyl derivatives of trimethylene carbonate; .delta.-valerolactone; .beta.-butyrolactone; .gamma.-butyrolactone; .epsilon.-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan-2-one; 2,5-diketomorpholine; pivalolactone; .alpha.,.alpha. diethylpropiolactone; ethylene carbonate; ethylene oxalate; 3-methyl-1,4-dioxane-2,5-dione; 3,3-diethyl-1,4-dioxan-2,5-dione-; 6,6-dimethyl-dioxepan-2-one; 6,8-dioxabicycloctane-7-one and polymer blends thereof. Additional exemplary polymer or polymer blends include, by non-limiting example, a polydioxanone, a polyhydroxybutyrate-co-hydrox-yvalerate, polyorthocarbonate, a polyaminocarbonate, and a polytrimethylene carbonate. Aliphatic polyesters used in the present invention can be homopolymers or copolymers (random, block, segmented, tapered blocks, graft, triblock, etc.) having a linear, branched or star structure. Poly(iminocarbonates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly(ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol. 30(1), page 498, 1989 by Cohn (e.g., PEO/PLA). Polyalkylene oxalates, for the purpose of this invention, include those described in U.S. Pat. Nos. 4,208,511; 4,141,087; 4,130,639; 4,140,678; 4,105,034; and 4,205,399. Polyphosphazenes, co-, ter- and higher order mixed monomer based polymers made from L-lactide, D,L-lactide, lactic acid, glycolide, glycolic acid, para-dioxanone, trimethylene carbonate and E-caprolactone such as are described by Allcock in The Encyclopedia of Polymer Science, Vol. 13, pages 31-41, Wiley Intersciences, John Wiley & Sons, 1988 and by Vandorpe, et al in the Handbook of Biodegradable Polymers, edited by Domb, et al., Hardwood Academic Press, pp. 161-182 (1997). Polyanhydrides include those derived from diacids of the form HOOC—C.sub.6H.sub.4—O—(—CH.sub.2).sub.m—O—C.sub.6H.sub.4—COOH, where “m” is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150. Polyorthoesters such as those described by Heller in Handbook of Biodegradable Polymers, edited by Domb, et al., Hardwood Academic Press, pp. 99-118 (1997).

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

What is claimed is:

1. A suture anchor system comprising:
a distal anchor body having a suture attachment and a guide wire trailing proximally from the distal anchor body; and
a proximal anchor body having a central bore passing therethrough sized to accommodate the guide wire, and one or more purchase enhancements extending from the proximal anchor body.

2. A suture anchor system according to claim 1 and further comprising a suture passer comprising an elongated flexible member threaded through the suture attachment and having a distal suture capture configuration.

3. A suture anchor system according to claim 2 wherein the suture capture configuration comprises a loop through which suture can be passed.

4. A suture anchor system according to claim 1 and further comprising a first driver having an elongated driver body with the distal anchor body attached to a distal end thereof and the guide wire extending along the driver body.

5. A suture anchor system according to claim 1 wherein the one or more purchase enhancements comprise screw threads.

6. A suture anchor system comprising:
a distal anchor body having a suture attachment and a distal awl tip, the distal anchor body being mounted on an awl driver whereby it can be driven into a bone to create a bone hole; and
a proximal anchor body sized to fit into a bone hole created by the distal anchor body and having one or more purchase enhancements extending from the proximal anchor body.

7. A suture anchor system according to claim 6 and further comprising a suture passer comprising an elongated flexible member threaded through the suture attachment and having a distal suture capture configuration.

8. A suture anchor system according to claim 7 wherein the suture capture configuration comprises a loop through which suture can be passed.

9. A suture anchor system according to claim 6 wherein the purchase enhancements comprise screw threads.

10. A method for attaching a soft tissue to a bone, the method comprising the steps of:
implanting into the bone a distal suture anchor body, leaving a guide wire extending proximally out of the bone from the distal suture anchor body;
threading a suture from the soft tissue through a suture attachment on the distal suture anchor body, the suture extending from the soft tissue to the distal suture anchor body and out of the bone when the distal suture anchor body is implanted into the bone; and
implanting over the guide wire and into the bone a proximal suture anchor body such that at least a portion of the suture is trapped between the proximal suture anchor body and the bone.

11. A method according to claim 10 and further comprising the step of threading the suture through the suture attachment on the distal body by placing the suture into a suture capture section of a suture shuttle threaded through the suture attachment and then pulling the suture shuttle through the suture attachment to draw the suture capture section and thus the suture through the suture attachment.

12. A method according to claim 11 wherein the suture shuttle is positioned through the suture attachment during the step of implanting the distal anchor body.

13. A method according to claim 10 wherein the proximal anchor body is threaded and the step of implanting the proximal anchor body comprises threading it into the bone behind the distal suture body.

14. A method according to claim 10 wherein a desired tension is applied to the suture after implanting the distal anchor body and prior to locking the suture by fully implanting the proximal anchor body.

15. A method for attaching a soft tissue to a bone, the method comprising the steps of:
piercing a bone and creating a bone hole with a distal suture anchor body which has a distal awl tip and a suture attachment with a length of suture passing therethrough, the distal suture anchor body being mounted on an awl driver;
removing the awl driver and leaving the distal anchor body implanted into the bone hole with a first portion of the suture extending out of the bone hole;
threading a second portion of the suture through the soft tissue;
tensioning the suture by drawing on the first portion; and
implanting into the bone hole proximal of the distal anchor body a proximal suture anchor body such that at least a portion of the suture is trapped between the proximal suture anchor body and the bone.

16. A method according to claim 15 wherein the proximal anchor body is threaded and the step of implanting the proximal anchor body comprises threading it into the bone behind the distal suture body.
